(19) United States
(12) Patent Application Publication
(10) Pub. No.: US 2024/0023978 A1
(43) Pub. Date: Jan. 25, 2024

(54) Surgical Guide

(71) Applicant: Italo Lozada, Weston, FL (US)

(72) Inventor: Italo Lozada, Weston, FL (US)

(21) Appl. No.: 17/870,306

(22) Filed: Jul. 21, 2022

(51) Int. Cl. A61B 17/17; A61C 1/08; A61B 34/10

(52) U.S. Cl. CPC A61B 17/176; A61C 1/084; A61B 34/10

(57) ABSTRACT

A surgical template for the placement and installation of a dental prosthesis is provided. The surgical template comprises a framework connectable to a patient's mouth, a drilling template connectable to the framework, and a dental prosthesis connectable to the framework. The drilling template allows customized and guided drilling for placement of implants, and the dental prosthesis comprises openings which align with the drilling template guide holes, allowing directed placement of the implants through the dental prosthesis openings. In many embodiments, the implant is able to engage with both the bone of the patient and the dental prosthesis simultaneously, thereby anchoring the prosthesis to the bone of the patient.

### BACKGROUND OF THE INVENTION

### Field of the Invention

The present invention relates generally to dental prostheses. More particularly the present invention relates to a dental prosthesis guide system which is formed to guide the placement of implants to anchor a dental prosthesis such as a full denture, partial denture, or crown.

### Description of Related Art

Generally, the installation of a dental prosthesis replicating a tooth is performed in two steps. The first step is the installation of a bone screw into a bone of a patient. This involves drilling a hole, and then setting a screw. This screw must then set for a period of time to allow the patient's body to adapt and accept the screw, and allow new bone growth in and around the screw. Only after this time period has elapsed can a replica tooth prosthesis (commonly called a “crown”) be installed by connecting it to this now secure screw. Usually this connection is via an adhesive. This process requires a substantial amount of a dentist's time, leading to higher costs. Further, the waiting period after the screw is implanted is uncomfortable and difficult for the patient. Further still, the bone target for screw installation, which will be drilled, is often not uniform, and is difficult to estimate. As such, the drilling may easily be off, causing a misaligned screw, or worse.

Moreover, unguided drilling creates a number of additional difficulties such as maintaining proper drill orientation throughout the entire drilling process. Failure to do so can cause unwanted widening of the drilled hole as well as a non-straight and non-uniform hole.

Therefore, what is needed is a dental prosthesis guide system that may allow the prosthesis to be installed at the same time as the installation of the implant, and that may also aid in guiding proper placement of the implant.

### SUMMARY OF THE INVENTION

The subject matter of this application may involve, in some cases, interrelated products, alternative solutions to a particular problem, and/or a plurality of different uses of a single system or article.

In one aspect, a surgical guide is provided. This surgical guide may be useful for placement of a dental prosthesis in the mouth of a patient. The surgical guide has a framework connectable to the bone in the mouth of a patient. The frame work has a plurality of anchor pin holes each sized to receive an anchor pin therethrough to connect the framework to a bone of a patient. The framework further has a plurality of connectors operable to connect one of a drilling template and a dental prosthesis. The surgical guide further includes a drilling template which has a plurality of guide apertures to guide a drill bit. The drilling template is removably connectable to the framework which properly positions the guide apertures for drilling into the patient's bone for the later placement of implants. The surgical guide further includes a dental prosthesis which is connectable to the framework as well. This prosthesis is formed to replace the tooth or teeth missing from the patient's mouth. The dental prosthesis has a plurality of implant guide holes. Each implant guide hole is sized and operable to allow a portion of an implant to pass through the implant guide hole, and sized and operable to allow a portion of the implant to engage with the dental prosthesis.

In another aspect, a method of installation of a dental prosthesis is provided. The method involves drilling a plurality of holes in a bone of a patient's mouth using a drill and then placing an anchor pin through an anchor pin hole of a framework into one of the plurality of drilled holes, thereby holding the framework in place to a mouth of the patient. The method further involves connecting a drilling template to the framework, the drilling template comprising a plurality of guide apertures to guide a drill for proper placement of drilled holes in a patient's mouth for the placement of dental implants. The method involves drilling an implant hole into the patient's mouth through each of the plurality of guide apertures, and then removing the drilling template from the framework. A dental prosthesis may then be connected to the framework. This dental prosthesis has a plurality of implant guide holes, each implant guide hole aligned with the implant hole drilled into the patient's mouth using the drilling template. An implant may be installed through the implant guide hole of the prosthesis. The installing step comprises setting a part of the implant into the implant hole, while a second different part of the implant engages with the dental prosthesis, thereby anchoring the prosthesis to the bone of the patient.

### BRIEF DESCRIPTION OF THE DRAWINGS

FIG. 1 provides a flow a bottom perspective view of the surgical guide base framework and drilling template.

FIG. 2 provides a view of the framework positioned in a mouth of a patient.

FIG. 3 provides a view of another embodiment of the prosthesis and framework.

FIG. 4 provides a view of another embodiment of the prosthesis and framework.

FIG. 5 provides a view of still another embodiment of the drilling template and framework.

FIG. 6 provides a view of still another embodiment of the framework and prosthesis.

FIG. 7 provides a view of yet another embodiment of the framework and prosthesis.

FIG. 8 provides a view of an embodiment of the surgical guide.

FIG. 9 provides a view of an embodiment of the prosthesis and a plurality of sleeves fittable into the prosthesis.

FIG. 10 provides a view of another embodiment of a sleeve contemplated herein.

FIG. 11 provides a view of still another embodiment of performing an osteotomy using the drilling template of the surgical guide before placement of the implants.

### DETAILED DESCRIPTION

The detailed description set forth below in connection with the appended drawings is intended as a description of presently preferred embodiments of the invention and does not represent the only forms in which the present invention may be constructed and/or utilized. The description sets forth the functions and the sequence of steps for constructing and operating the invention in connection with the illustrated embodiments.

Generally, the present invention concerns a surgical guide for placement of a dental prosthesis configured to be installed concurrently with an implant (bone screw) to support and anchor the prosthesis. Further, the invention concerns a dental prosthesis that may also include a guide for a drill to ensure proper orientation of drilling a hole and or placing an implant in bone. The guide hole of the prosthesis may align with the surgical guide and may be formed to align the drilled hole and/or implant with the ideal bone installation area. Moreover, by directly attaching the implant to the prosthesis, no adhesive cement is needed, thereby eliminating problems caused by the cement including gum irritation and infection.

The dental prosthesis contemplated herein is formed to replicate a tooth it is replacing. In one embodiment, this may be a single tooth. In another embodiment, the prosthesis may cover multiple teeth. For example, the technology used herein may be used for a single tooth replacement (a “crown”) or multiple teeth including complete arch restorations, for example full dentures, hybrid bridges, partial denture, or fixed bridge, and the like. In addition to this, the prosthesis is formed having a guide hole through its height that will allow the implant to be placed therethrough. In some embodiments, a drill bit or self-drilling screw can pass through the guide hole into the bone. Once the drill bit drills a proper hole as guided by the guide hole, a screw may be inserted, anchoring the prosthesis to the bone. This guide hole may be threaded, or smooth, depending on embodiment. In one threaded embodiment, the threads may be formed directly into the prosthesis material. In another embodiment, a sleeve may contain the threads and may have the prosthesis formed around it. The sleeve may be, for example, a durable material such as titanium or the like.

As is known in the art, an implant (screw) preferably may be given a period of time to be accepted by the patient's body before it can fully support the dental prostheses (often referred to as a crown). This is because the patient's body takes time to accept the implanted screw, and grow bone around it to hold it securely in place.

The screw contemplated herein may be any shaft, threaded or otherwise, capable of being implanted securely into a bone of a patient. The screw may be configured to set into a pre-drilled hole, may be self-driven (also called “self-drilling”), or self tapping, among other options.

In one embodiment, to accommodate for this, allowing the present invention to be installed concurrently with the screw, the surgical guide as disclosed herein will be used. The surgical guide base is positioned securely on the bone of the patient and can hold the prosthesis and/or surgical guide cover in proper position. In many embodiments, the surgical guide cover connects to the surgical guide base and is sized to fit over the dental prosthesis which may or may not be connected to the surgical guide base, depending on embodiment.

In further embodiments, once the implants are placed along with the prosthesis, an additional securing component may be used to secure the prosthesis while the implant is setting. In one embodiment, wings may be used to secure the prosthesis to adjacent teeth. In another embodiment, embodiment allowing securing of the prosthesis while the screw is setting involves a retainer. The retainer may be removably connectable to adjacent existing teeth in the patient's mouth, or another component in the patient's mouth such as a surgical guide or other base, jaws, adjacent bone, and the like, and may protect and secure the prosthesis.

In embodiments wherein a tooth is being extracted and will soon after be replaced with a prosthesis, a 3D model may be taken of the tooth, and then replicated such that the replacement prosthesis is a nearly identical replica of the original removed tooth. For example a 3D scan of a removed tooth or teeth of the patient may be taken using a computerized system. Based on this scan, the prosthesis may be generated automatically using a computer controlled system based on the scanned removed tooth. This generated replica may be formed as a crown only, or may comprise a partial or complete root for the prosthesis.

In embodiments wherein the tooth has been removed and the original tooth or teeth are not available, a best guess may be made to replicate the original based on measurements of the installation area.

In one embodiment, a method of identifying the best area for bone drilling may include taking a 3D X-ray of the jaw or bone around the tooth. This will provide a 3D model of the bone structure around the area to be drilled, and will allow the operator to identify the path the screw will take. Based on this, the tooth may be fabricated with the guide hole to guide the drill into the bone accordingly. However, it should be understood that any method to identify the best path for the screw may be used. It should be understood that the prosthesis guide hole may be configured based on the identified best area for drilling to control angle, width, and depth, thereby limiting damage and danger that can be done when drilling and/or setting the screw.

Typically, after the prosthesis and screw have been installed in place, the guide hole may be filled, thereby completing the prosthesis. The filling may be performed with any filling known in the art such as a composite, dental metal, or the like. In another embodiment, the filling may be performed by applying a veneer, covering the hole. This embodiment may be particularly useful when the guide hole protrudes through a side of the prosthesis.

The dental prosthesis may be made of any material capable of being mounted within a mouth of the patient. Generally, the prosthesis may be any material typically used to make crowns, dentures, partial dentures, and similar prostheses in the dental field. However, it should be understood that this invention is not limited to these materials.

Similarly, the implant (also referred to as a screw) may be any material capable of being screwed into bone and set therein. Generally, the screw may be made of typical medical or dental implant material such as zirconium, titanium, or the like. However, it should be understood that this invention is not limited to these materials.

The drill contemplated herein may be any drill capable of drilling into bone tissue. Any existing dental or medical drill may be used, as well as future innovations on such drilling devices.

Generally, the surgical guide contemplated herein includes a base formed as a framework. The framework is specially formed to fit in a particular patient's mouth after taking measurements of the patient. The framework is then connected to the patient using, for example, pins which are placed into the bone of the patient's mouth after a hole is drilled to accommodate the pin. The surgical guide framework has an adequate number of anchor pin holes placed in strategic positions and orientations to secure the surgical guide base frame work in the correct position. During surgery, maximum attention should be paid to secure the surgical guide in the correct position in the patient's mouth and that it does not move in any direction from the correct position while being manipulated with the instruments. Proper location of the surgical guide frame work is important because the base frame work provides a foundation for the drilling template as well as the foundation for the prosthesis and defines the correct location for both.

A drilling template is removably connectable to the base framework. The drilling template defines a plurality of implant guide apertures which guide placement of implants in the proper location and orientation. Determination of the proper implant position is based on measurements and analysis of a particular patient and will vary from patient to patient. In one embodiment, a plurality of tabs on the drilling template connect to a plurality of corresponding surgical guide holes in the frame work. The connection of the drilling template to the framework is done in a specific and fixed way, to ensure that the drilling template is in just the right position. The apertures of the drilling template allow the soft tissue punch and osteotomy to be performed using a drill or drills in just the right position and orientation. In one embodiment, the drilling template has a convex outer face extending away from the mouth of the patient. This defines a space between template and user mouth so that, in some embodiments, existing teeth can remain in place without interference.

During the formation and design of the drilling template for the patient, bone quality and quantity was considered in the implant planning. The sequence, speed and length of the drilling is indicated in a personalized surgical protocol provided with the surgical template. The surgical guide also may be provided with one or a plurality of necessary stoppers to make the depths of the holes correctly. These stoppers prevent a drill bit from going too deep into the tissue of the patient. The stopper or stoppers may be formed into the drilling template, or may be a separate tool to position against the drilling template and through which the drill bit passes. Once a shoulder on the drill meets the stopper, the proper depth of drilling has been reached.

Once the osteotomies have been made using the drilling template by following the personalized protocol, the prosthesis is attached to the base framework. Like the drilling template, the prosthesis is specifically formed and sized to be properly positioned for the patient based on the connection with the framework. In one embodiment, a plurality of tabs on the prosthesis connect to a plurality of corresponding surgical guide holes in the frame work. These tabs may be removed once the prosthesis is permanently placed and fixed in position. The connection of the prosthesis to the framework is done in a specific and fixed way, to ensure that the prosthesis is in just the right position.

Once the prosthesis is in place, the implants are seated into the openings formed in the bone. The prosthesis comprises a plurality of apertures which are designed to align with the openings formed into the bone. The prosthesis is formed so as to be placed at the same time as the placement of the implants. As discussed above, this allows for much quicker and more efficient placement of dental prosthesis, rather than the prior art system of placing implants, waiting for them to form into the bone, and then attaching the prosthesis.

Accordingly, the prosthesis is connected to the framework base, and known to be properly positioned. Implants, such as threaded implants may then be installed through the prosthesis apertures into the bone of the user. The implant is operable to engage with both the bone of the patient and the prosthesis so as to connect one to the other. In one embodiment, a head or abutment of the implant may engage with a shoulder formed within the prosthesis aperture to anchor the two together. In another embodiment, an abutment of the implant may have threads on an outer surface. These threads may engage corresponding threads on an inside of the aperture of the prosthesis and/or a threaded sleeve positioned within the aperture of the prosthesis. Any structure that allows the implant to engage both the bone of the patient and the prosthesis to join them together may be used without straying from the scope of this invention. The engaging of both the bone and the prosthesis allows for immediate fixation of the prosthesis to the bone.

When the implants and prosthesis are secured in place, the tabs or other connectors on the prosthesis are cut off or otherwise removed from the prosthesis. The framework is no longer needed, and thus the pins securing it to the bone are removed, and the framework is removed. Thus the prosthesis is fully installed and operational. Any holes or openings in the prosthesis may be filled with a suitable material such as resin or acrylic as is known in the art.

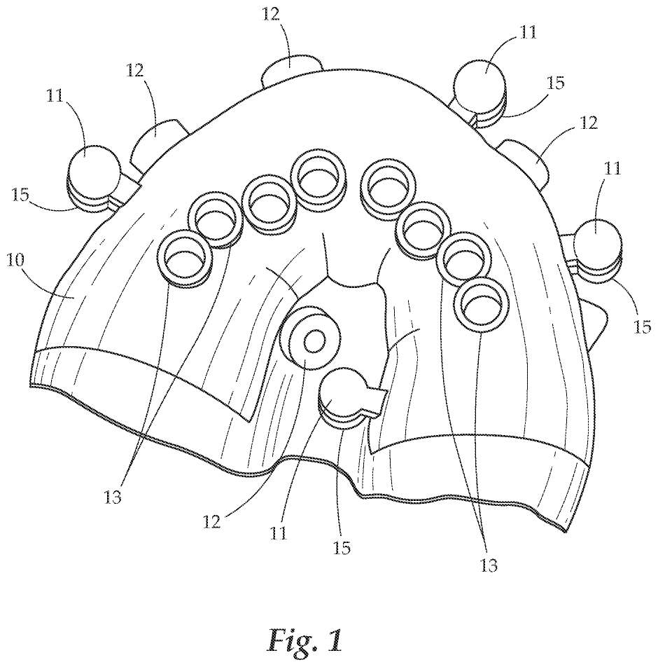

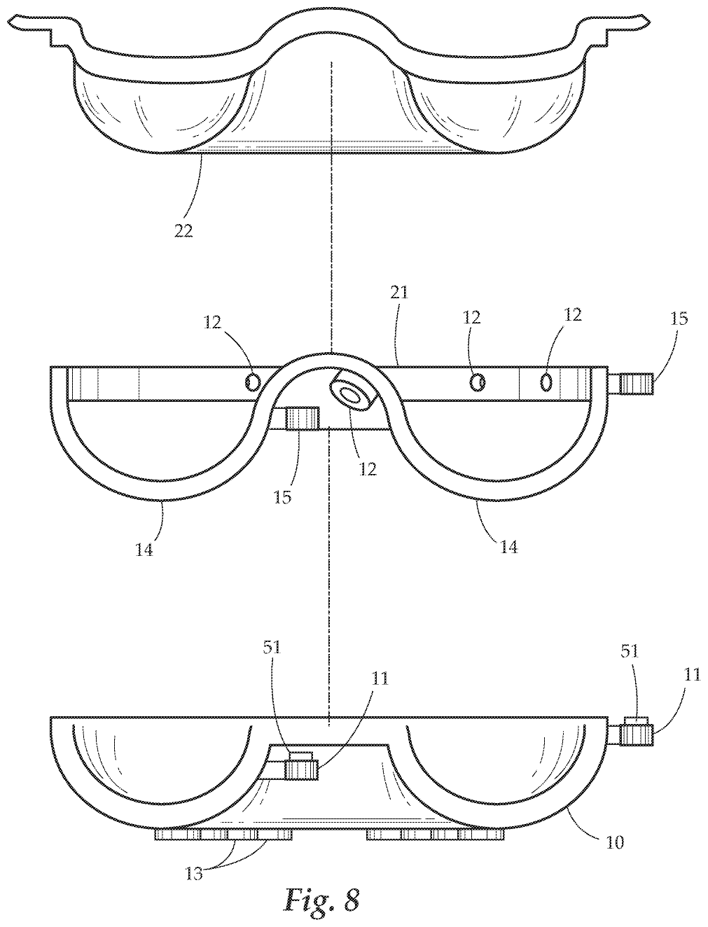

Turning now to FIG. 1, a bottom perspective view of the surgical guide base framework and drilling template is shown. The base framework has a rear portion 14 which is in a rear of the mouth behind the rear of the drilling template 10, as well as a perimeter side (not visible in this figure). The framework has a plurality of anchor pin holes 12 which align with predetermined areas of the patient's mouth suitable for placement of pins (not shown) into the bone of the patient, thereby holding the framework in place. The framework also defines a plurality of surgical guide tab holes 15 which allow tab connectors 11 of the drilling template to removably connect to the framework. The tab connectors 11 of this embodiment are sized to frictionally fit within the surgical guide tab holes 15 to hold the drilling template 10 in the proper position in the patient's mouth. The drilling template 10 comprises a plurality of guide apertures 13 which are sized and oriented to guide a drill of an operator to form holes into the bone of the patient. The drilling template 10 is specially formed based on measurements of the patient to cause the drilling holes to be optimally located. These holes may then receive an implant therein to anchor the prosthesis to the patient. Different views of the framework and drilling template positioned in the patient's mouth can be seen in FIG. 8

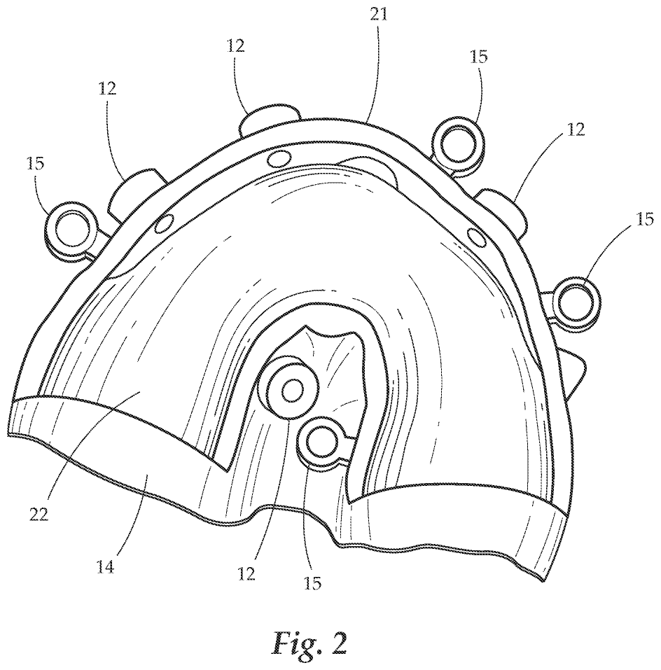

FIG. 2 shows a view of the framework positioned in a mouth of a patient. Here, the framework 21 is used for a full denture and all teeth are removed from the gums 22. The framework 21 defines the area to receive the surgical guide and/or prosthesis (not shown). A plurality of anchor pin holes 12 are placed around the outside and inside of the framework. These anchor pin holes 12 are sized and positioned to receive anchor pins which anchor the framework 21 to the patient's bone, securing it in place and in turn allowing the drilling template and prosthesis to be secured in place via their connection to the framework 21. The framework also has a plurality of guide tab holes 15 which extend away from the framework and allow connection of corresponding tabs which extend away from the drilling template and prosthesis. In the embodiment shown, the anchor pin holes 12 have an extending spacer flange to provide additional structural support and proper depth placement of the anchor pins. The anchor pin holes extend generally perpendicularly to the sidewalls of the framework 21. The guide tab holes 15 are extended away from the sidewalls of the framework 21 by an arm. The guide tab holes 15 extend generally parallel to the sidewall of the framework in an up and down direction. With the framework attached to the bone above or below the intended location of the prosthesis (depending on if top or bottom prosthesis), the guide tab holes 15 are oriented to allow connection of the drilling template/prosthesis above or below the framework 21.

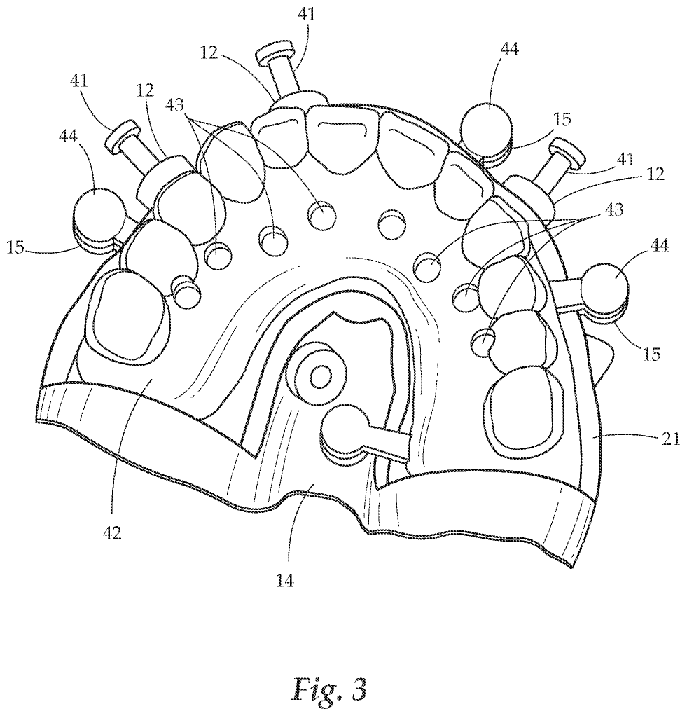

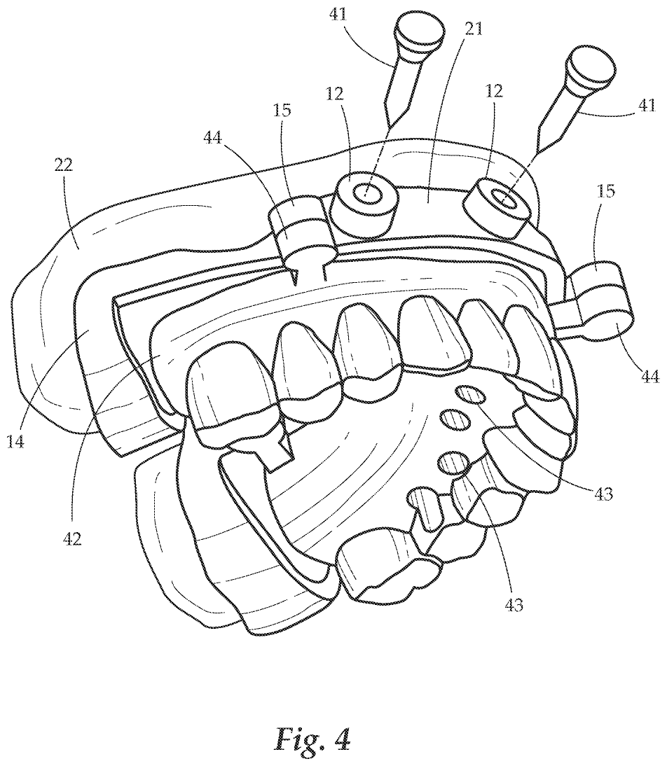

FIGS. 3 and 4 provide views of the prosthesis attached to the framework during a step of placing the implants through the prosthesis to anchor the prosthesis to the patient's bone. The prosthesis 42, shown here as a full denture, has a plurality of guide tabs 44 which extend away from the prosthesis 42. The tabs (not seen in this view) extend upwardly towards a top of the prosthesis 42 i.e. away from the teeth and into the guide tab holes 15. Anchor pins 41 can be seen placed through anchor pin holes 12 into the bone of the patient. The prosthesis further comprises a plurality of implant guide holes 43. The implant guide holes 43 are positioned to align with the drilling template guide apertures 13. A dental implant (not shown) fits through the implant guide hole 43 and into the hole drilled into the patient as guided by the drilling template guide apertures 13. The implant guide hole 43 is sized and oriented to guide the implant properly to align with the hole drilled into the patient, and also sized and oriented to allow the implant to engage with the prosthesis 42. As such, when placed, the implant will engage both the bone of the patient as well as the prosthesis 42, holding the two together for immediate placement of the prosthesis.

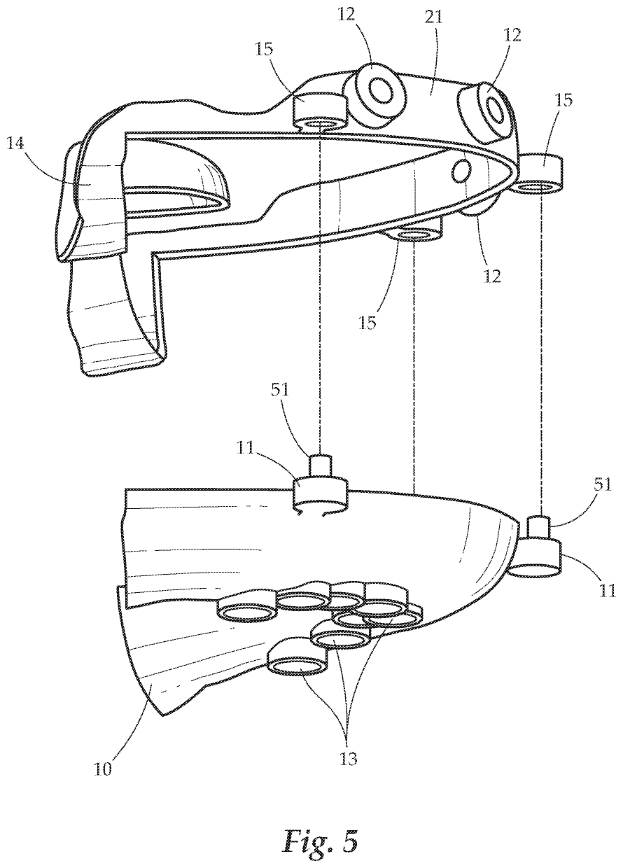

FIG. 5 provides a view of the connection of the drilling template 10 to the framework 21. As can be seen, tabs 51 extend upward and fit into the opening of the guide tab holes 15 to frictionally engage therein. In one embodiment, the tab 51 may have an annular ridge extending around its perimeter to add a “snap” fit function. However, it should be understood that in varying embodiments, the connection between framework and surgical guide/prosthesis may vary without straying from the scope of this invention. In many embodiments, the drilling template and prosthesis may have the same structure operable to connect to the same portion of the framework 21, however in other embodiments, different structure may be used to connect the drilling template to the framework and prosthesis to the framework.

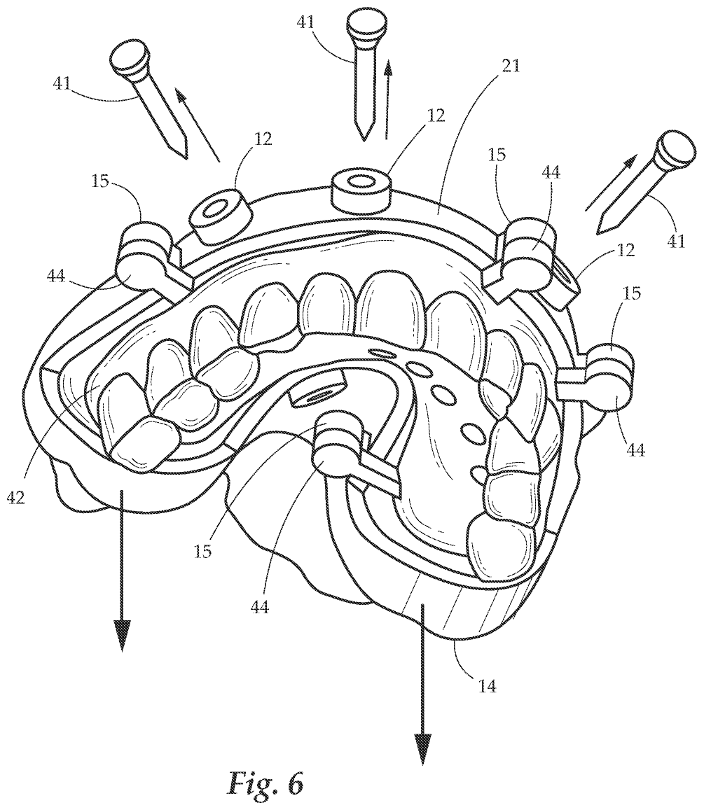

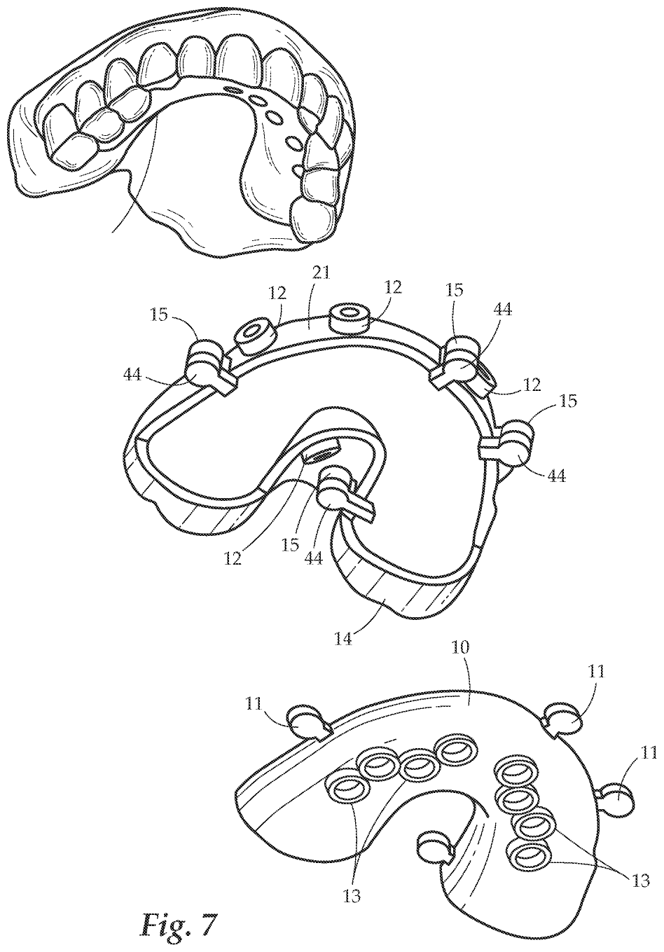

FIG. 6 and shows views of the procedure to remove the framework after the prosthesis has been positioned and set. Once the implants have been placed to join the prosthesis 42 to the bone of the patient, the framework 21 may be removed. This may be done soon after placing the implants, or a time may be allowed to pass to allow the patient's bone to grown into the implants, further securing them in place. In either case, the guide tabs 44 are cut away from the prosthesis 42. This disconnects the framework 21 from the prosthesis 42. The anchor pins 41 are removed through anchor pin holes 12. This separates the framework 21 from the patient. As such, the framework 21 may then be removed, leaving the prosthesis 42 installed. As seen in FIG. 7, the prosthesis 42 remains in place, while framework 21 and drilling template 10 are removed.

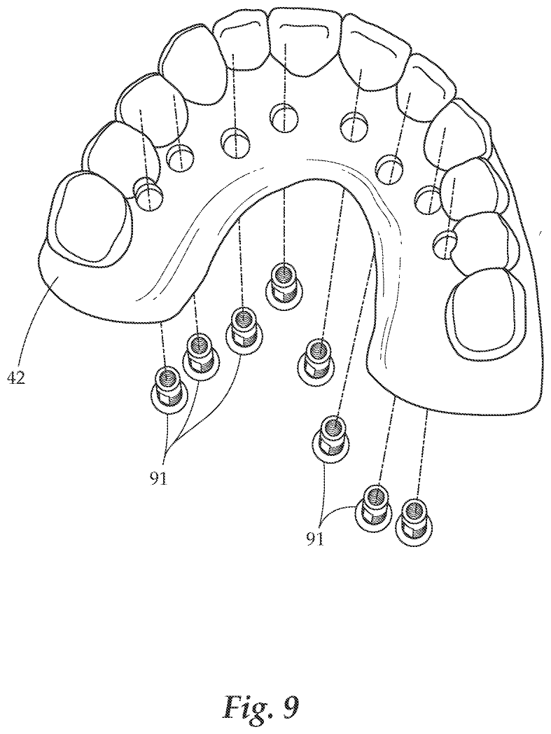

FIG. 9 shows a view of a plurality of threaded sleeves which fit within the implant guide holes 43 of the prosthesis 42. When used in certain embodiments, the threaded sleeves 91 are able engage with threads on the implant or a part of the implant such as an abutment having threads on its outer surface. These sleeves may be fixedly connected to the inside of the prosthesis 42, or may be frictionally fit into the guide holes, adhered therein, or seated on a shoulder or other structure or connection that prevents them from passing through the implant guide holes 43.

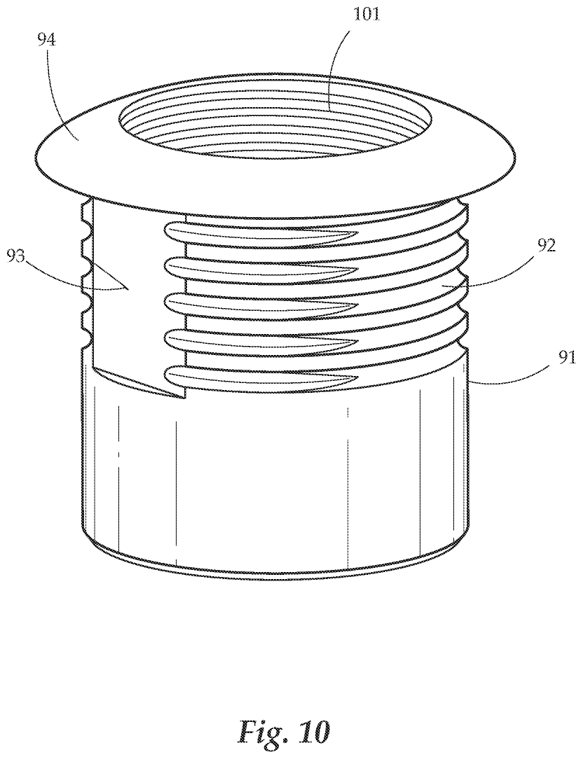

FIG. 10 shows a view of the sleeve 91 and the threads 101 on the inside. These threads 101 are configured to engage with a portion of the implant such as an abutment on the implant or the implant threads itself. In another embodiment, the sleeve interior may be smooth but have a shoulder or taper which mechanically engages a part of the implant. The sleeve 91 further includes grooves 92 which provide additional surface area for adhesive to join the sleeve to the prosthesis 42 within guide holes 43. Further, the sleeve 91 has a flat face 93 which engages with a corresponding shape within guide hole 43 and prevents rotation and ensures proper orientation. Flanged head 94 ensures that the sleeve seats at the proper depth and configuration within the guide hole 43.

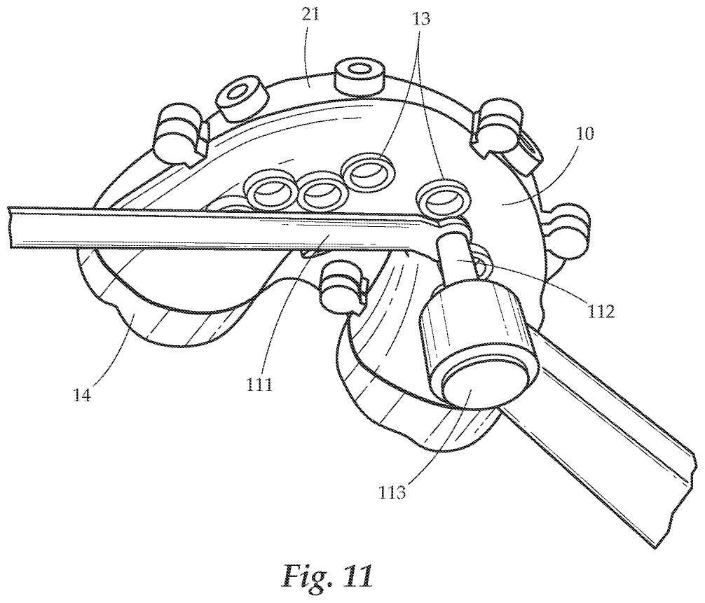

FIG. 11 shows a view of an embodiment of performing an osteotomy using the drilling template of the surgical guide before placement of the implants. The framework 21 is attached to the mouth of the patient, and the drilling template 10 is attached thereto. A drill 113 having a bit 112 is guided through the guide aperture 13. The guide aperture 13 controls location and angle/direction of the drill bit but in some embodiments, not necessarily depth. In the embodiment shown, a spacer 111 is provided which fits between the shoulder of the drill 113 and the surface of the guide aperture 13. This spacer 111, which may vary in thickness, prevents the drill bit 112 from extending too far into the patient's bone. Given a known length of the bit 112, the spacer allows control of an exact depth of the drill bit 112. In the embodiment shown, the spacer 111 is formed as an elongate bar having a small aperture through which the drill bit 112 passes. Opposite sides of the spacer will abut the drill 113 and the drilling template 10 when the drill bit is at its deepest point.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

What is claimed is:

1. A surgical guide for placement of a dental prosthesis comprising:
a framework comprising a plurality of anchor pin holes each sized to receive an anchor pin therethrough to connect the framework to a bone of a patient, and the framework further comprising a plurality of connectors operable to connect one of a drilling template and a dental prosthesis;
the drilling template comprising a plurality of guide apertures to guide a drill bit, the drilling template removably connectable to the framework;
the dental prosthesis connectable to the framework, the dental prosthesis comprising a plurality of implant guide holes, each implant guide hole sized and operable to allow a portion of an implant to pass through the implant guide hole, and sized and operable to allow a portion of the implant to engage with the dental prosthesis.

2. The surgical guide of claim 1 wherein the dental prosthesis further comprising a threaded sleeve positioned in each of the plurality of implant guide holes, the threaded sleeve having threads on an inside of the sleeve configured to engage with a thread on an outer surface of at least part of the implant.

3. The surgical guide of claim 1 wherein the drilling template having a concave shape facing the surgical guide.

4. The surgical guide of claim 1 wherein the plurality of connectors of the framework each comprises a guide tab hole, and wherein the drilling template comprises a plurality of guide tabs, each of the plurality of guide tabs configured to fit within the guide tab hole, and wherein the dental prosthesis comprises a plurality of guide tabs, each of the plurality of guide tabs configured to fit within the guide tab hole.

5. The surgical guide of claim 1 wherein the plurality of connectors of the framework each comprises a guide tab hole, and wherein the drilling template comprises a plurality of guide tabs, each of the plurality of guide tabs configured to fit within the guide tab hole.

6. The surgical guide of claim 1 wherein the plurality of connectors of the framework each comprises a guide tab hole, and wherein the dental prosthesis comprises a plurality of guide tabs, each of the plurality of guide tabs configured to fit within the guide tab hole.

7. The surgical guide of claim 4 wherein each guide tab hole extends away from a sidewall of the framework and is oriented to receive the guide tab in a direction from bottom to top of the framework.

8. The surgical guide of claim 4 wherein each guide tab of the drilling template and each guide tab of the dental prosthesis is extended away from a perimeter side of the drilling template or prosthesis by an arm, and wherein the guide tab is oriented upwardly towards a top of the drilling template or prosthesis.

9. The surgical guide of claim 1 wherein an interior of each of the plurality of implant guide holes has a threaded surface configured to engage with a thread on an outer surface of at least part of the implant.

10. The surgical guide of claim 1 wherein an interior of each of the plurality of implant guide holes comprises a shoulder or tapered surface to engage with the implant.

11. The surgical guide of claim 1 wherein the drilling template is connected to the framework.

12. The surgical guide of claim 1 wherein the dental prosthesis is connected to the framework.

13. The surgical guide of claim 12 wherein the dental prosthesis further comprises a plurality of implants, each of the plurality of implants positioned in one of the plurality of implant guide holes, and each of the plurality of implants having a portion which is engaged with the dental prosthesis limiting a movement of the implant through its corresponding implant guide hole.

14. The surgical guide of claim 1 wherein the dental prosthesis further comprises a plurality of implants, each of the plurality of implants positioned in one of the plurality of implant guide holes, and each of the plurality of implants having a portion which is engaged with the dental prosthesis limiting a movement of the implant through its corresponding implant guide hole.

15. A method of installation of a dental prosthesis comprising the steps of:
drilling a plurality of holes in a bone of a patient's mouth using a drill;
placing an anchor pin through an anchor pin hole of a framework into one of the plurality of drilled holes, thereby holding the framework in place;
connecting a drilling template to the framework, the drilling template comprising a plurality of guide apertures;
drilling an implant hole into the patient's mouth through each of the plurality of guide apertures;
removing the drilling template;
connecting a dental prosthesis to the framework, the dental prosthesis comprising a plurality of implant guide holes, each implant guide hole aligned with the implant hole drilled into the patient's mouth using the drilling template;
installing an implant through the implant guide hole of the prosthesis, wherein the installing step comprises setting a part of the implant into the implant hole, while a second different part of the implant engages with the dental prosthesis, thereby anchoring the prosthesis to the bone of the patient.

16. The method of claim 15 further comprising the steps of cutting a plurality of tabs off the dental prosthesis, the plurality of tabs connected to the framework.

17. The method of claim 15 wherein the installing step comprises engaging a thread on an exterior of the implant with a threaded insert positioned within the implant guide hole of the prosthesis.

18. The method of claim 15 wherein the installing step comprises engaging a portion of the implant with a shoulder or taper within the implant guide holes.

19. The method of claim 15 wherein the framework comprises a plurality guide tab holes to connect the drilling template, and wherein the drilling template comprises a plurality of guide tabs, each of the plurality of guide tabs configured to fit within the guide tab hole, the step of connecting the drilling template to the framework comprising fitting the plurality of guide tabs into the plurality of guide tab holes.

20. The method of claim 15 wherein the framework comprises a plurality guide tab holes to connect the dental prosthesis, and wherein the dental prosthesis comprises a plurality of guide tabs, each of the plurality of guide tabs configured to fit within the guide tab hole, the step of connecting the prosthesis to the framework comprising fitting the plurality of guide tabs into the plurality of guide tab holes.
